(19) United States
(12) Patent Application Publication
(10) Pub. No.: US 2022/0031191 A1
(43) Pub. Date: Feb. 3, 2022

(54) CONTACTLESS BREATHING DETECTION METHOD AND SYSTEM THEREOF

(71) Applicant: NATIONAL YUNLIN UNIVERSITY OF SCIENCE AND TECHNOLOGY, Yunlin County (TW)

(72) Inventors: Chuan-Yu CHANG, Yunlin County (TW); Min-Hsiang CHANG, Yunlin County (TW)

(21) Appl. No.: 17/026,288

(22) Filed: Sep. 20, 2020

(30) Foreign Application Priority Data
Jul. 30, 2020 (TW) 109125868

(51) Int. Cl. A61B 5/08; A61B 5/1171; A61B 5/00

(52) U.S. Cl. CPC A61B 5/0816; A61B 5/7257; A61B 5/0077; A61B 5/1176

(57) ABSTRACT

A contactless breathing detection method is for detecting a breathing rate of a subject. The contactless breathing detection method includes a photographing step, a capturing step, a calculating step, and a converting step. The photographing step is performed to provide a camera to photograph the subject to generate a facial image. The capturing step is performed to provide a processor module to capture the facial image to generate a plurality of feature points. The calculating step is performed to drive the processor module to calculate the feature points according to an optical flow algorithm to generate a plurality of breathing signals. The converting step is performed to drive the processor module to convert the breathing signals to generate a plurality of power spectrums, respectively. The processor module generates an index value by calculating the power spectrums, and the breathing rate is extrapolated from the index value.

### RELATED APPLICATIONS

This application claims priority to Taiwan Application Serial Number 109125868, filed Jul. 30, 2020, which is herein incorporated by reference.

### BACKGROUND

### Technical Field

The present disclosure relates to a breathing detection method and a system thereof. More particularly, the present disclosure relates to a contactless breathing detection method and a system thereof.

### Description of Related Art

A breathing detection is a very important part in much clinical detection. The deviation of breathing rate or the depth of breathing can be regarded as important indicators for judging whether the human body is healthy. The conventional breathing detection mainly has the following three methods: the air flow of nose and mouth, the variation of chest impedance, and the up and down movement of the chest cavity. However, each of the aforementioned three methods is a contact sensing device and is connected to the host device by wires. The contact sensing device is not easy to use or wear and also makes subjects feeling uncomfortable, so that the breathing rate is less measured or paid attention to.

In view of this, how to develop a contactless breathing detection system for the problems of the above-mentioned breathing detecting device becomes the goal of the public and relevant industry efforts.

### SUMMARY

According to an embodiment of a methodical aspect of the present disclosure, a contactless breathing detection method is for detecting a breathing rate of a subject and includes a photographing step, a capturing step, a calculating step and a converting step. The photographing step is performed to provide a camera to photograph the subject to generate a facial image. The capturing step is performed to provide a processor module to capture the facial image to generate a plurality of feature points. The calculating step is performed to drive the processor module to calculate the feature points according to an optical flow algorithm to generate a plurality of breathing signals. The converting step is performed to drive the processor module to convert the breathing signals to generate a plurality of power spectrums, respectively. The processor module generates an index value by calculating the power spectrums, and the breathing rate is extrapolated from the index value.

According to an embodiment of a structural aspect of the present disclosure, a contactless breathing detection system is for detecting a breathing rate of a subject and includes a camera and a processor module. The camera photographs the subject to generate a facial image. The processor module is electrically connected to the camera and receives the facial image. The processor module includes a capturing sub-module, a calculating sub-module and a converting sub-module. The capturing sub-module captures the facial image to generate a plurality of feature points. The calculating sub-module is connected to the capturing sub-module and receives the feature points. The calculating sub-module calculates the feature points according to an optical flow algorithm to generate a plurality of breathing signals. The converting sub-module is connected to the calculating sub-module and receives the breathing signals. The converting sub-module converts the breathing signals to generate a plurality of power spectrums, respectively. The converting sub-module generates an index value by calculating the power spectrums, and the breathing rate is extrapolated from the index value.

### BRIEF DESCRIPTION OF THE DRAWINGS

The present disclosure can be more fully understood by reading the following detailed description of the embodiment, with reference made to the accompanying drawings as follows:

FIG. 1 shows a block diagram of a contactless breathing detection system according to an embodiment of a structural aspect of the present disclosure.

FIG. 2A shows a schematic view of a plurality of feature points of the contactless breathing detection system of FIG. 1.

FIG. 2B shows another schematic view of a plurality of feature points of the contactless breathing detection system of FIG. 1.

FIG. 3 shows a block diagram of the contactless breathing detection system according to an embodiment of another structural aspect of the present disclosure.

FIG. 4 shows a flow chart of a contactless breathing detection method according to an embodiment of a methodical aspect of the present disclosure.

FIG. 5 shows a flow chart of a calculating step of the contactless breathing detection method of FIG. 4.

FIG. 6 shows a flow chart of a converting step of the contactless breathing detection method of FIG. 4.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

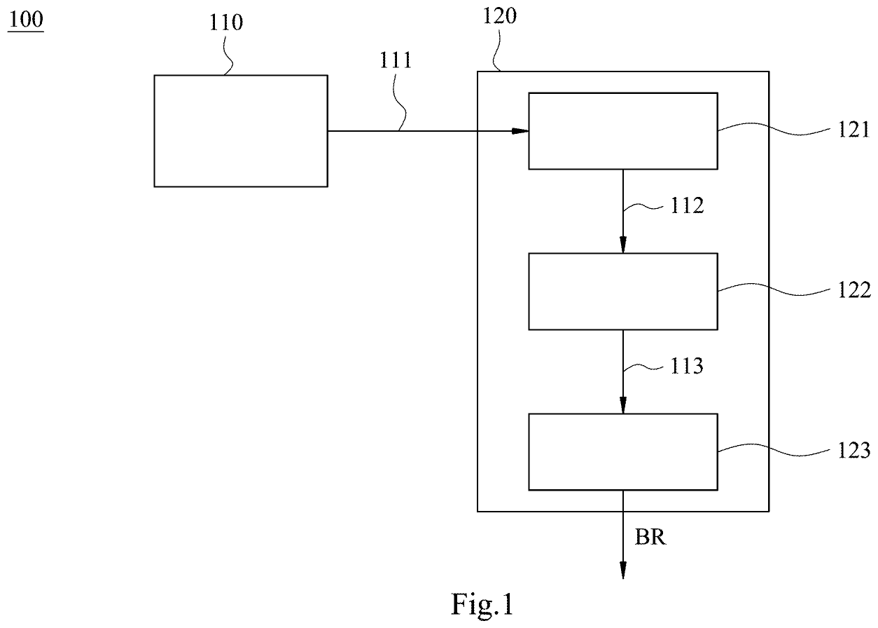

FIG. 1 shows a block diagram of a contactless breathing detection system 100 according to an embodiment of a structural aspect of the present disclosure. In FIG. 1, the contactless breathing detection system 100 is for detecting a breathing rate BR of a subject and includes a camera 110 and a processor module 120. In the embodiment, the camera 110 can be a video camera, a mobile phone or a video recording device, and the processor module 120 can be a computer, but the present disclosure is not limited thereto.

The camera 110 is for photographing a face in front view of the subject to generate a facial image 111. The processor module 120 is electrically connected to the camera 110 and receives the facial image 111. The processor module 120 includes a capturing sub-module 121, a calculating sub-module 122 and a converting sub-module 123. The capturing sub-module 121 captures the facial image 111 to generate a plurality of feature points 112. The calculating sub-module 122 is connected to the capturing sub-module 121 and receives the feature points 112. The calculating sub-module 122 calculates the feature points 112 according to an optical flow algorithm (e.g., Lucas-Kanade method) to generate a plurality of breathing signals 113. The converting sub-module 123 is connected to the calculating sub-module 122 and receives the breathing signals 113. The converting sub-module 123 converts the breathing signals 113 to generate a plurality of power spectrums, respectively. The converting sub-module 123 generates an index value by calculating the power spectrums, and the breathing rate BR is extrapolated from the index value.

Therefore, the present disclosure tracks the feature points 112 of the facial image 111 by the optical flow algorithm and converts the feature points 112 into the power spectrums, and then the breathing rate BR is extrapolated from the index value of a maximum peak of each of the power spectrums, so that the contactless breathing detection system 100 can take a contactless way to measure the breathing rate BR of the subject.

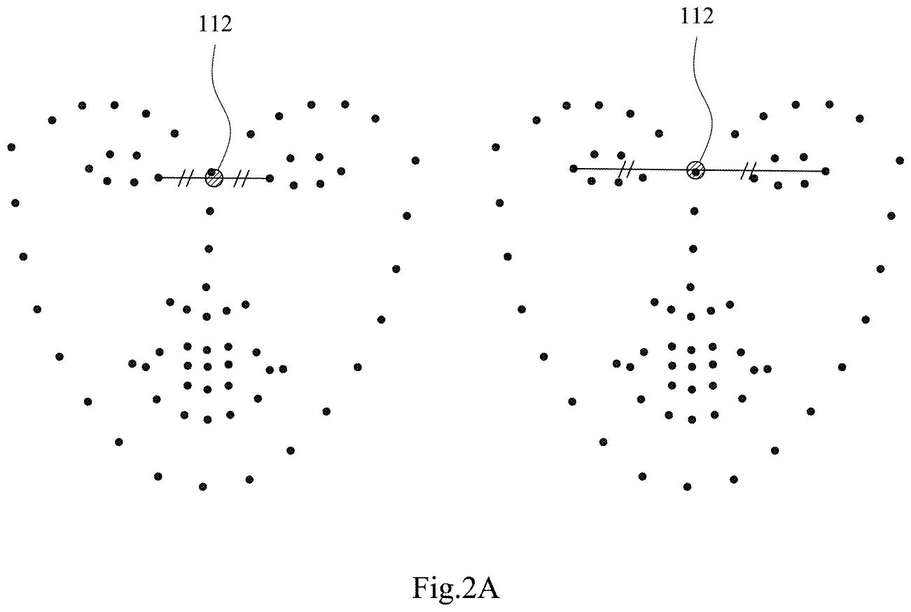

Please refer to FIGS. 1, 2A and 2B. FIG. 2A shows a schematic view of a plurality of feature points 112 of the contactless breathing detection system 100 of FIG. 1. FIG. 2B shows another schematic view of a plurality of feature points 112 of the contactless breathing detection system 100 of FIG. 1. In FIGS. 2A and 2B, the capturing sub-module 121 captures the facial image 111 and finds 68 points on the facial image 111. The capturing sub-module 121 captures the 68 points to obtain 7 feature points 112 which are less affected by external factors. In FIG. 2A, the feature point 112 of a left face can be a midpoint of an inner eye corner, and the feature point 112 of a right face can be a midpoint of an outer eye corner. In FIG. 2B, the feature point 112 of the left face can be a midpoint of an inner-outer right eye corner, and the feature points 112 of the right face can be a nose-root point, a nose-tip point, a nose-base point and a lower jaw point. It is hereby stated that the present disclosure is not limited to the feature points 112 described above.

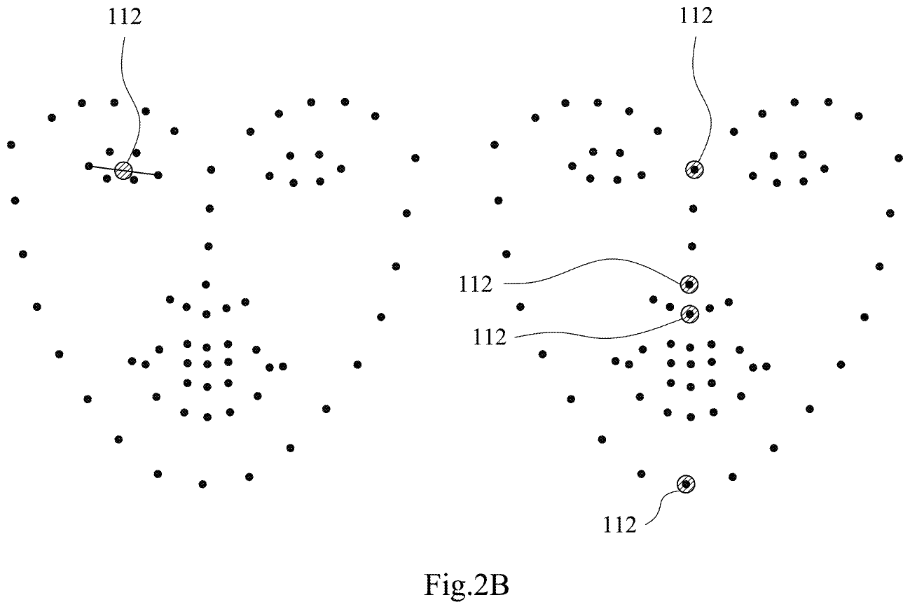

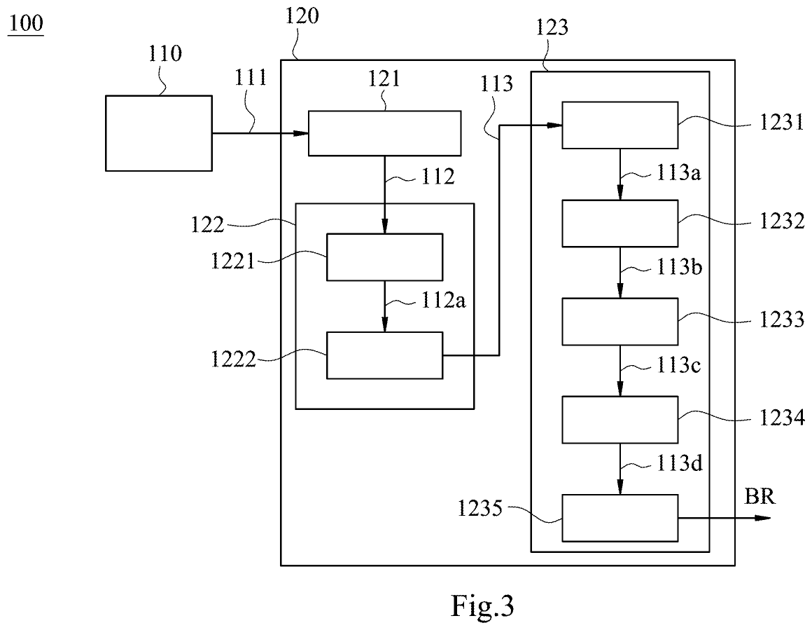

Please refer to FIGS. 1, 3 and 4. FIG. 3 shows a block diagram of the contactless breathing detection system 100 according to an embodiment of another structural aspect of the present disclosure. FIG. 4 shows a flow chart of a contactless breathing detection method S100 according to an embodiment of a methodical aspect of the present disclosure. In the embodiment of FIG. 3, the arrangement of the camera 110, the capturing sub-module 121, the calculating sub-module 122 and the converting sub-module 123 of the contactless breathing detection system 100 is the same as the corresponding components of the embodiment in FIG. 1, and will not be detailedly described herein.

In FIG. 3, the calculating sub-module 122 can includes an optical flow unit 1221 and an analyzing unit 1222. The optical flow unit 1221 receives the feature points 112 and tracks the variation of the feature points 112 caused by the difference between the previous frame and the next frame with the optical flow algorithm. The optical flow unit 1221 outputs a mixed signal 112a. The analyzing unit 1222 is connected to the optical flow unit 1221 and receives the mixed signal 112a. The analyzing unit 1222 separates the mixed signal 112a through an Independent Component Analysis (ICA) to obtain seven separated breathing signals 113. Furthermore, the converting sub-module 123 can include a Fourier transform unit 1231, a filtering unit 1232, a power converting unit 1233, an index calculating unit 1234 and a breathing rate calculating unit 1235. The Fourier transform unit 1231 performs a Fast Fourier transform on the breathing signals 113 to generate a plurality of frequency domain signals 113a. The filtering unit 1232 is connected to the Fourier transform unit 1231 and receives the frequency domain signals 113a. The filtering unit 1232 filters out each of frequency domain signals 113b having the frequency between 0.15 Hz and 0.35 Hz from the frequency domain signals 113a. The power converting unit 1233 is connected to the filtering unit 1232 and receives the frequency domain signal 113b. The power converting unit 1233 processes the frequency domain signals 113b to generate a plurality of power spectrums 113c, respectively. The index calculating unit 1234 is connected to the power converting unit 1233 and generates an index value 113d according to the power spectrums 113c. The breathing rate calculating unit 1235 is connected to the index calculating unit 1234 and extrapolates the breathing rate BR according to the index value 113d.

In FIG. 4, the contactless breathing detection method S100 can be applied to the contactless breathing detection system 100 shown in FIGS. 1 and 3. The contactless breathing detection method S100 is for detecting the breathing rate BR of the subject. The contactless breathing detection method S100 includes a photographing step S110, a capturing step S120, a calculating step S130 and a converting step S140. The photographing step S110 includes providing the camera 110 to photograph the subject to generate the facial image 111. The capturing step S120 includes providing the processor module 120 to capture the facial image 111 to generate the feature points 112. The calculating step S130 includes driving the processor module 120 to calculate the feature points 112 according to the optical flow algorithm to generate the breathing signals 113. The converting step S140 includes driving the processor module 120 to convert each of the breathing signals 113 to generate the power spectrums 113c, respectively. The processor module 120 generates the index value 113d by calculating the power spectrums 113c, and the breathing rate BR is extrapolated from the index value 113d.

Therefore, the present disclosure captures the feature points 112 of the facial image 111 with the processor module 120 and tracks the variation of the feature points 112 with the optical flow algorithm. Finally, the present disclosure finds the index value 113d so as to estimate the breathing rate BR of the subject.

In detail, the contactless breathing detection method S100 can be divided into two stages: the first stage includes the image capture of the face and the capture of the feature points 112 (that is, the photographing step S110 and the capturing step S120); the second stage includes the calculation and the conversion of the breathing rate BR (that is, the calculating step S130 and the converting step S140).

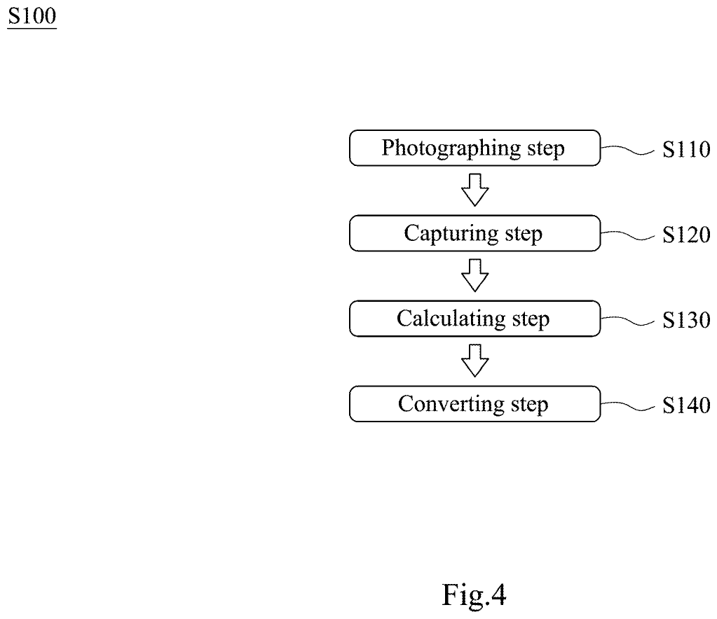

Please refer to FIGS. 3 and 5. FIG. 5 shows a flow chart of the calculating step S130 of the contactless breathing detection method S100 of FIG. 4. In FIG. 5, the calculating step S130 includes a tracking step S131 and an analyzing step S132. The tracking step S131 includes executing the optical flow algorithm through the optical flow unit 1221 and tracks the feature points 112 to generate the mixed signal 112a. Particularly, the calculating sub-module 122 can include the optical flow unit 1221. The optical flow unit 1221 executes the optical flow algorithm and includes a displacement, a X-coordinate of each of the feature points 112, a Y-coordinate of each of the feature points 112, a time parameter and the mixed signal 112a, the displacement is expressed as Dᵢ, and the X-coordinate is expressed as X_{Fi}(t), the Y-coordinate is expressed as Y_{Fi}(t), the time parameter is expressed as t, and the mixed signal 112a is expressed as S and conforms to a following formula (1):

$$\{ \begin{matrix}{D}_{i} = \sqrt{{[ {X}_{Fi} ( t ) - {X}_{Fi} ( t - 1 ) ]}^{2} - {[ {Y}_{Fi} ( t ) - {Y}_{Fi} ( t - 1 ) ]}^{2}} , i = 1 , 2 , … , n \\ S = {[ {D}_{1} , {D}_{2} , … , {D}_{n} ]}^{T}\end{matrix} . \qquad ( 1 )$$

In detail, in the tracking step S131, a total of seven facial feature points 112 are extracted, and n=7 in the formula (1). The optical flow unit 1221 finds the variation (i.e., the displacement Dᵢ) of the seven feature points 112 caused by the difference between the previous frame and the next frame in the time sequence with the tracking characteristics of the optical flow algorithm to obtain the mixed signal 112a. The mixed signal 112a can be a variety of different signals which include the signals of the body motion, the heart rate, and the breathing rate BR.

Successively, the analyzing step S132 processes the mixed signal 112a to generate the breathing signal 113 with the analysis unit 1222. Particularly, in order to further find out the frequency band matching the breathing rate BR, the analysis unit 1222 separates the mixed signal 112a through the ICA to obtain the seven separated breathing signals 113. In detail, because the human head (or face) contains many subtle movements, it is necessary to calculate the displacement Dᵢ to obtain the mixed signal 112a. Then, according to the principle of blind signal separation, the ICA is used for preliminary separation to decompose the independent signal sources hidden in the mixed signal 112a to select the signals matching the breathing rate BR.

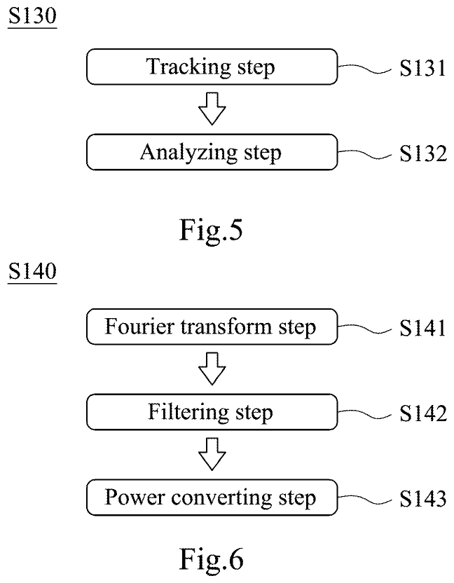

Please refer to FIGS. 3 and 6. FIG. 6 shows a flow chart of the converting step S140 of the contactless breathing detection method S100 of FIG. 4. In FIG. 6, the converting step S140 includes a Fourier transform step S141, a filtering step S142 and a power converting step S143. The Fourier transform step S141 includes providing the Fourier transform unit 1231 to process each of the breathing signals 113 to generate the frequency domain signals 113a, respectively. In detail, the Fourier transform unit 1231 transforms each of the breathing signals 113 into the corresponding frequency domain signals 113a with the Fast Fourier Transform (FFT), and the FFT is a linear integral transform which is for transforming signals between the time domain and the frequency domain.

Furthermore, each of the frequency domain signals 113a can have a corresponding frequency. The filtering step S142 includes providing the filtering unit 1232 to filter out each of the frequency domain signals 113b having the frequency between 0.15 Hz and 0.35 Hz. The filtering unit 1232 can be a Butterworth Filter which filters out the interesting section from the frequency domain signals 113a. Since the frequency of the breathing is between 0.15 Hz and 0.35 Hz, the filtering unit 1232 is for filtering out frequencies other than the range between 0.15 Hz and 0.35 Hz, and the remained frequency domain signals 113b is the interesting section.

Moreover, the power converting step S143 includes processing the frequency domain signals 113b through the power converting unit 1233 to generate the power spectrums 113c, respectively. In detail, according to Fourier analysis, anyone of the physical signals can be decomposed into a discrete or continuous spectrum. The total energy of the signal in a limited period of time is limited, so that the power spectrums 113c can be calculated by the above characteristic. The calculation of the power spectrums 113c is that after the signal is subjected to the FFT, the real square and imaginary square of the frequency domain signal 113b are added together to obtain the power spectrums 113c.

More detail, the converting sub-module 123 can include the power converting unit 1233, an index calculating unit 1234 and a breathing rate calculating unit 1235. The power converting unit 1233 includes a power, a real part, a variable, and an imaginary part, the power is expressed as P₁, the real part is expressed as Rᵢ, the variable is expressed as u, and the imaginary part is expressed as Iᵢ and conforms to a following formula (2):

Pᵢ(u)=Rᵢ²(u)+Iᵢ²(u), i=1,2, . . . ,n (2).

Successively, in the converting step S140, a maximum power and an average power are extrapolated from the power spectrums 113c by the index calculating unit 1234. The maximum power minuses the average power, and the channel with the largest result is selected as the index value 113d for calculating the breathing rate BR, and then importing the index value 113d into the breathing rate calculating unit 1235, and using the formula (4) of the breathing rate BR to obtain the breathing rate BR of the final subject and conforming to the following formulas (3) and (4):

$$\begin{matrix}\{ \begin{matrix}α = argmax ( {P}_{i}^{max} - {P}_{i}^{avg} ) \\ I = argmax ( {P}_{a} ( u ) )\end{matrix} . & ( 3 ) \\ Breathing Rate = 60 \times I . & ( 4 )\end{matrix}$$

The index value 113d is expressed as I, the breathing rate BR is expressed as Breathing Rate, Pᵢ^{max} is expressed as the maximum power, Pᵢ^{avg} is expressed as the average power, argmax is expressed as a function, and the function argma can find the value of the variation when the formula reaches the maximum, a is expressed as the maximum power Pᵢ^{max} and the average power Pᵢ^{avg} of the aforementioned value, and u is expressed as a variation.

In summary, the present disclosure has the following advantages: First, the breathing rate of the subject can be measured in the contactless way. Second, there is no need to use a contact-type wearing device so as to reduce the cost of the detecting device.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A contactless breathing detection method for detecting a breathing rate of a subject, the contactless breathing detection method comprising:
performing a photographing step to provide a camera to photograph the subject to generate a facial image;
performing a capturing step to provide a processor module to capture the facial image to generate a plurality of feature points;
performing a calculating step to drive the processor module to calculate the feature points according to an optical flow algorithm to generate a plurality of breathing signals; and
performing a converting step to drive the processor module to convert the breathing signals to generate a plurality of power spectrums, respectively, wherein the processor module generates an index value by calculating the power spectrums, and the breathing rate is extrapolated from the index value.

2. The contactless breathing detection method of claim 1, wherein a number of the feature points is 7, and the feature points are a midpoint of an inner eye corner, a midpoint of an outer eye corner, a midpoint of an inner-outer right eye corner, a nose-root point, a nose-tip point, a nose-base point and a lower jaw point, respectively.

3. The contactless breathing detection method of claim 1, wherein the calculating step comprises:
performing a tracking step to execute the optical flow algorithm through an optical flow unit and tracking the feature points to generate a mixed signal; and
performing an analyzing step to process the mixed signal with an analyzing unit to generate the breathing signals.

4. The contactless breathing detection method of claim 3, wherein the optical flow unit comprises a displacement, a X-coordinate of each of the feature points, a Y-coordinate of each of the feature points, a time parameter and the mixed signal, the displacement is expressed as Dᵢ, and the X-coordinate is expressed as X_{Fi}(t), the Y-coordinate is expressed as Y_{Fi}, the time parameter is expressed as t, and the mixed signal is expressed as S and conforms to a following formula:
$$\{ \begin{matrix}{D}_{i} = \sqrt{{[ {X}_{Fi} ( t ) - {X}_{Fi} ( t - 1 ) ]}^{2} - {[ {Y}_{Fi} ( t ) - {Y}_{Fi} ( t - 1 ) ]}^{2}} , i = 1 , 2 , … , n \\ S = {[ {D}_{1} , {D}_{2} , … , {D}_{n} ]}^{T}\end{matrix} .$$

5. The contactless breathing detection method of claim 1, wherein the converting step comprises:
performing a Fourier transform step to provide a Fourier transform unit to process the breathing signals to generate a plurality of frequency domain signals, respectively; and
performing a power converting step to process the frequency domain signals through a power converting unit to generate the power spectrums.

6. The contactless breathing detection method of claim 5, wherein the power converting unit comprises a power, a real part, a variable, and an imaginary part, the power is expressed as Pᵢ, the real part is expressed as Rᵢ, the variable is expressed as u, and the imaginary part is expressed as Iᵢ and conforms to a following formula:
Pᵢ(u)=Rᵢ²(u)+Iᵢ²(u), i=1,2, . . . ,n.

7. The contactless breathing detection method of claim 5, wherein each of the frequency domain signals has a frequency, and the converting step further comprises:
performing a filtering step to provide a filtering unit to filter out each of the frequency domain signals having the frequency between 0.15 Hz and 0.35 Hz.

8. A contactless breathing detection system for detecting a breathing rate of a subject, the contactless breathing detection system comprising:
a camera photographing the subject to generate a facial image; and
a processor module electrically connected to the camera and receiving the facial image, wherein the processor module comprises:
a capturing sub-module capturing the facial image to generate a plurality of feature points;
a calculating sub-module connected to the capturing sub-module and receiving the feature points, wherein the calculating sub-module calculates the feature points according to an optical flow algorithm to generate a plurality of breathing signals; and
a converting sub-module connected to the calculating sub-module and receiving the breathing signals, wherein the converting sub-module converts the breathing signals to generate a plurality of power spectrums, respectively, the converting sub-module generates an index value by calculating the power spectrums, and the breathing rate is extrapolated from the index value.

9. The contactless breathing detection system of claim 8, wherein the calculating sub-module comprises an optical flow unit, the optical flow unit executes the optical flow algorithm and comprises a displacement, a X-coordinate of each of the feature points, a Y-coordinate of each of the feature points, a time parameter and the mixed signal, the displacement is expressed as Dᵢ, and the X-coordinate is expressed as X_{Fi}(t), the Y-coordinate is expressed as Y_{Fi}(t), the time parameter is expressed as t, and the mixed signal is expressed as S and conforms to a following formula:
$$\{ \begin{matrix}{D}_{i} = \sqrt{{[ {X}_{Fi} ( t ) - {X}_{Fi} ( t - 1 ) ]}^{2} - {[ {Y}_{Fi} ( t ) - {Y}_{Fi} ( t - 1 ) ]}^{2}} , i = 1 , 2 , … , n \\ S = {[ {D}_{1} , {D}_{2} , … , {D}_{n} ]}^{T}\end{matrix} .$$

10. The contactless breathing detection system of claim 8, wherein the converting sub-module comprises a power converting unit, the power converting unit comprises a power, a real part, a variable, and an imaginary part, the power is expressed as Pᵢ, the real part is expressed as Rᵢ, the variable is expressed as u, and the imaginary part is expressed as Iᵢ and conforms to a following formula:
Pᵢ(u)=Rᵢ²(u)+Iᵢ²(u), i=1,2, . . . ,n.
